(19) United States
(12) Patent Application Publication
(10) Pub. No.: US 2007/0190162 A1
(43) Pub. Date: Aug. 16, 2007

(54) Colloidal suspension of submicronic particles for delivering active principles and method for preparing same

(76) Inventors: Sylvain Caillol, Paris (FR); Remi Meyruiex, Lyon (FR); Nathan Bryson, Toronto (CA); Alain Soum, Pessac (FR); Gerard Soula, Meyzieu (FR); Anne-Francoise Mingotaud, Toulouse (FR)

Correspondence Address: PATTON BOGGS LLP, 8484 WESTPARK DRIVE, SUITE 900, MCLEAN, VA, 22102, US

(21) Appl. No.: 11/583,941

(22) Filed: Oct. 20, 2006

Related U.S. Application Data
(63) Continuation of application No. 11/335,732, filed on Jan. 20, 2006.
Application No. 11/335,732 is a continuation of application No. 10/512,435, now abandoned.

(30) Foreign Application Priority Data
Apr. 23, 2003 (FR) PCT/FR03/01278
Apr. 26, 2002 (FR) 02 05312

(51) Int. Cl. A61K 9/14

(52) U.S. Cl. 424/499; 424/489

(57) ABSTRACT

The present invention is directed to a suspension of particles for delivering active principles, in particular proteins. Said particles are based on a diblock copolymer consisting of a neutral hydrophobic alpha hydroxy carboxylic acid polymer block and a hydrophilic linear polyaminoacid block with peptide alpha chaining, at least partly ionized. Said alpha hydroxy carboxylic acid polymer/linear polyaminoacid delivery particles spontaneously obtainable in the absence of surfactant can be stable. Said delivery particles are capable of being associated undissolved in colloidal suspension with at least an active principle and of delayed or prolonged release thereof. The invention is also directed to a powdery solid from which are derived the delivery particles and the preparation of said solid and said delivery particle suspension.

The field of the present invention is that of delivery particles (DPs), which can be used for the administration of active principles (APs). The latter are preferably medicinal products or nutrients for administration to an animal or human organism via the oral, nasal, vaginal, ocular, subcutaneous, intravenous, intramuscular, intradermal, intraperitoneal, intracerebral, parenteral, etc. route. In terms of chemical nature, the APs with which the invention is more particularly, but with no implied limitation, concerned are hydrophilic or amphiphilic, for example proteins, glycoproteins, peptides, polysaccharides, lipopolysaccharides, polynucleotides and organic molecules.

The present invention relates more specifically to colloidal suspensions of delivery particles, advantageously of submicronic type, based on hydrophobic polymer blocks and on hydrophilic polyamino acid blocks, of the polyGlu type.

The present invention is directed toward both naked particles per se, and the AP delivery systems consisting of the particles loaded with the AP(s).

The present invention also relates to pulverulent solids comprising these DPs. The invention also relates to processes for preparing said colloidal suspensions of particles, loaded with AP.

The aim of encapsulating APs in DPs is in particular to modify their duration of action and/or to convey them to the site of treatment and/or to increase the bioavailablility of said APs. Many encapsulation techniques have already been proposed. Such techniques are directed, firstly, toward enabling the AP to be transported to its site of therapeutic action, while at the same time protecting it against the body's attacks (hydrolysis, enzymatic digestion, etc.) and, secondly, toward controlling the release of the AP over its site of action, in order to maintain the amount available to the organism at the desired level. The APs with which these vicissitudes of delivery and residence in the body are concerned are, for example, proteins, but may also be any other products, organic molecules of synthetic or natural origin. The review by M. J. Humphrey (Delivery system for peptide drugs, edited by S. Davis and L. Illum, Plenum Press, N.Y. 1986) gives an account of the problem concerning the improvement of the bioavailability of APs and the advantage of systems for delivery and controlled release.

Among all the materials that can be envisioned for forming DPs, polymers are increasingly used on account of their intrinsic properties. As regards the list of specifications that it is desired to obtain for DPs, it is particularly demanding and comprises, in particular, the following specifications.
1 The first desired specification would be that the DPs would advantageously be able to form, without the aid of organic solvent and/or surfactant, a stable aqueous suspension.
2 It is desirable for it to be possible to obtain the DPs and the DP-AP systems by means of a process which does not denature the AP.
3 Another desired specification would be for the polymer which constitutes the DPs to be biocompatible, to be able to be eliminated (by excretion) and/or to be biodegradable and, better still, would be for it to be metabolized into products that are not toxic for the organism. In addition, the biodegradation in the organism should take a sufficiently short period of time.
4 It would also be desirable for the DPs to be sufficiently small in size for them to be able to undergo, in suspension in a liquid, a sterilizing filtration by means of a filter for which the pore diameter is less than or equal to 0.2 μm.
5 The DPs should advantageously make it possible to control the rate of release of the AP.
6 Another important specification would be for the DP-AP systems to be able to constitute excellent injectable medicinal products. This improved ability to be administered by injection—e.g. intravenous, subcutaneous or intramuscular—“injectability”, is characterized by:
   (i) a reduced volume injected (for a given therapeutic dose),
   (ii) a low viscosity.
7 The two properties are satisfied when the therapeutic dose of AP is combined with a minimum amount of DP. In other words, the DPs should have a high degree of loading with AP.
8 The cost specific to the DPs in an injectable preparation should be low, and here again, the DPs should have a high degree of loading with AP. In fact, the small size and a high degree of loading are major specifications desired for the DPs.
It is also advantageous for the polymer constituting the DPs not to induce an immune response.
9 For the family of hydrophilic and amphiphilic APs, in particular proteins, it would be advisable to have DPs which are adapted to this family of APs in terms of ease of association and of release, and in terms of nondenaturing character.

The prior technical propositions, described above, have attempted to satisfy this set of specifications. By way of illustration, mention will be made of prior propositions (a) to (j):
(a) Patent U.S. Pat. No. 5,286,495 relates to a process of encapsulation by spraying proteins in aqueous phase, using materials having opposite charges, namely: alginate (negatively charged) and polylysine (positively charged). This manufacturing process makes it possible to produce particles greater than 35 μm in size.
(b) In addition, emulsion techniques are commonly used for preparing microparticles loaded with AP. For example, patent applications WO 91/06286, WO 91/06287 and WO 89/08449 disclose such emulsion techniques in which use is made of organic solvents in order to solubilize polymers, for example of the polylactic type. However, the solvents have proven to be possibly denaturing, in particular for peptide or polypeptide APs.
(c) Biocompatible DPs called protenoids, described as early as 1970 by X. Fox and K. Dose in “Molecular Evolution and the origin of Life”, publisher Marcel Dekker Inc. (1977), are also known. Thus, patent application WO 88/01213 proposes a system based on a mixture of synthetic polypeptides whose solubility depends on the pH. To obtain the matricial microparticles according to that invention, the mixture of polypeptides is solubilized and then a change in pH causes proteinoid particles to precipitate. When the precipitation is carried out in the presence of an AP, this AP is encapsulated in the particle.
(d) Mention will also be made, as a matter of interest, of U.S. Pat. No. 4,351,337 which is the product of a field other than that of the delivery of the APs characteristic of the invention. That patent discloses implants of masses attached and located at quite precise sites in the body. These implants are hollow tubes or capsules of microscopic size (160 μm and of length equal to 2000 μm), consisting of copolymers of copoly(amino acids)—e.g. poly(glutamic acid-leucine) or poly(benzylglutamate-leucine)—obtained by copolymerization of monomers of N-carboxyanhydrides of amino acids (NCAs). An AP is included by means of a technique of evaporation of the solvent of a mixture of polymer and of AP. U.S. Pat. No. 4,450,150 belongs to the same family as U.S. Pat. No. 4,351,337 studied above and essentially has the same subject matter. The constituent PAAs are poly(glutamic acid-ethyl glutamate).
(e) PCT patent application WO 97/02810 discloses a composition for the controlled release of active principles, comprising a plurality of lamellar particles of a biodegradable polymer, at least partly crystalline (lactic acid polymer), and of an AP absorbed onto said particles. In this case, the release of the active principle takes place by desorption.
(f) The subject of PCT patent application WO 96/29991 is particles of polyamino acids that are useful for delivering AP, including in particular hydrophilic APs such as insulin. These particles are between 10 and 500 nm in size. The particles according to WO 96/29991 form spontaneously by bringing PAAs into contact with an aqueous solution. The PAAs comprise hydrophobic neutral amino acid, AAO, monomers and hydrophilic ionizable, AAI, monomers.
   These particles can be loaded with insulin, at best to an amount of 6.5% by dry weight of insulin relative to the mass of PAA. The degree of loading, Ta, is measured according to a procedure Ma described later.
(g) Patent application EP 0 583 955 (U.S. Pat. No. 5,449,513) discloses polymeric micelles capable of physically trapping hydrophobic APs. These micelles consist of block copolymers: PEG/polyAANO, AANO=Amino Acide Neutre hydrophObe=hydrophobic neutral amino acid. The AANO may be: Leu, Val, Phe, Bz-O-Glu, Bz-O-Asp, the latter being preferred. The hydrophobic active principles AP trapped in these PEG/polyAANO micelles are e.g.: adriamycin, indomethacin, daunomycin, methotrexate, mitomycin.
(h) U.S. Pat. No. 5,514,380 discloses a copolymer comprising a lactic acid polymer block and a poly(ethylene oxide) (PEG) block, that is useful as a matrix for the release of medicinal products. There is no mention of microparticles prepared from this copolymer.
(i) Many publications are, moreover, known which describe particles based on PEG/lactic acid polymer (LAP) copolymers, for the sustained release of active principles, including in particular:
   Biomaterials 17 (1996) 1575-1581, Vittaz et al,
   Polym. Adv. Technol. 10, 647-654 (1999), Kinn et al.
In the copoly(PEG)(LAP) particles, the AP is physically encapsulated in the LAP core by codissolution in an organic solvent for the AP and for the copoly(PEG) (LAP). It results from this that the APs made up of proteins could be difficult to encapsulate in these copoly(PEG)(LAP) particles since the risks of denaturation of the AP are considerable.
(j) The article Biomaterials 19 (1998) 1501-1505/K. E. Gonsalves et al. describes microparticles (diameter 200 nm) of poly(L-lactic)(Asp) copolymer and of poly(L-lactic)(Ser) copolymer. These copoly(LAP) (PAA) particles−PAA=PolyAmino Acid—are obtained in the form of an emulsion by mechanical agitation of a stabilizing (surfactant) aqueous solution of polyvinyl alcohol (PVA) and an organic solution (CH₂Cl₂) of copoly(LAP) (PAA). These hollow spherical particles are stabilized by means of the PVA surfactant, which forms an outer layer, the inner layer consisting of the copoly (LAP) (PAA). In order to exist, these particles require the use of the stabilizing PVA surfactant. There is no question of an at least partial ionization of the PAA. In addition, the authors estimate that these copoly(LAP)(PAA) particles could be used for the controlled release of medicinal products. This assessment is supported by no technical experiment. This article does not disclose a stable colloidal suspension comprising these microparticles, and even less, any ability of the latter to associate in colloidal suspension in undissolved form, with at least one active principle.

It emerges from the above that the prior technical propositions described above incompletely satisfy the list of specifications indicated above and, in particular, as regards the association of the particles with active principles (in particular proteins) and the ability of these AP-loaded particles to release said APs in vivo without them having been altered by the delivery.

Given this irrefutable fact, an essential objective is to be able to provide novel DPs which form spontaneously, and without the aid of surfactants, aqueous suspensions of DP that are stable (at physiological pHs) and suitable for delivering APs (in particular sensitive APs such as proteins).

Another essential objective of the present invention is to provide novel DPs in stable colloidal aqueous suspension or in pulverulent form and based on poly(amino acids) (PAAs), it being the duty of the novel DPs to satisfy as well as possible specifications 1 to 9 of the abovementioned specification list.

Another essential objective of the invention is to provide a novel suspension of DPs whose characteristics are completely controlled, in particular in terms of the degree of loading with AP and in terms of control of the kinetics of release of the AP.

Another essential objective of the present invention is to provide injectable medicinal suspensions. The specifications required for such suspensions are a small injection volume and a low viscosity. It is important for the mass of colloidal particles per injection dose to be as low as possible, without limiting the amount of active principle AP transported by these particles, so as not to harm the therapeutic efficacy.

Another essential objective of the invention is to provide an aqueous colloidal suspension or a pulverulent solid comprising particles for delivering active principles which satisfy the abovementioned specifications and which constitute an appropriate pharmaceutical form suitable for administration, for example oral administration, to humans or animals.

Another essential objective of the invention is to provide a colloidal suspension comprising particles for delivering active principles, which can be filtered through 0.2 μm filters for sterilization purposes.

Another essential objective of the invention is to propose a method for preparing particles (dry or in suspension in a liquid) of hydrophobic PAA/hydrophilic polymer blocks, that are useful in particular as vectors for active principles (in particular proteins such as insulin, IFN, IL-2, factor VIII, EPO, etc.), this method having to be simpler to implement, non-denaturing for the active principles, and also having to always allow a fine control of the mean particle size of the particles obtained.

Another essential object of the invention is the use of the abovementioned particles in aqueous suspension or in solid form for preparing medicinal products (e.g. vaccines), in particular for oral, nasal, vaginal, ocular, subcutaneous, intravenous, intramuscular, intradermal, intraperitoneal intracerebral or parenteral administration, it being possible for the hydrophilic active principles of these medicinal products to be in particular proteins, glycoproteins, peptides, polysaccharides, lipopolysaccharides, oligonucleotides and polynucleotides.

Another objective of the present invention is to provide a medicinal product, of the system for sustained release of active principles type, which is easy and economical to produce, and which is also biocompatible and able to provide a very high level of bioavailability of the AP.

The objectives relating to the products (among others) are achieved by means of the present invention which concerns, first of all, a colloidal suspension of submicronic particles which can be used in particular for delivering APs, these particles being individualized supramolecular arrangements based on an amphiphilic copolymer including:
at least one block of α-peptide-linked hydrophilic linear polyamino acid(s) (PAAs), the hydrophilic amino acids AAI constituting this PAA block being identical to or different from one another;
and at least one block of at least one hydrophobic polymer, made up of an α-hydroxy-carboxylic acid polymer (HCAP)— preferably lactic acid polymer (LAP) or glycolic acid polymer (GAP)-

characterized in that:
it can be obtained spontaneously in the absence of surfactant, by bringing together the amphiphilic copolymer and a liquid that is not a solvent for the AAIs;
it is stable even in the absence of surfactants;
the AAIs of the copolymer are at least partially in ionized form;
the particles are capable of associating in suspension in the nondissolved state with at least one AP and of releasing it, in particular in vivo, in a sustained and/or delayed manner.

One of the inventive bases of these novel delivery particles DPs, in colloidal aqueous suspension that is stable at physiological pHs or in the pulverulent solid state, comes from the original selection of a (hydrophobic α-hydroxycarboxylic acid polymer) (hydrophilic polyamino acid) block copolymer making it possible to obtain particles of submicronic size, which form a colloidal suspension (preferably aqueous) that is stable at all physiological pHs, in the absence of surfactants, which are adapted to all pHs.

The fact that these (HCAP) (PAA) microparticles have at least some of their AAIs in ionized form in suspension also constitutes an innovative characteristic.

Another notable advantage of these submicronic particles comes from their ability to allow the adsorption to their surface of APs, in colloidal suspension in the nondissolved state, and therefore in the absence of any aggressive organic solvent or surfactant. This type of association is to be distinguished from the processes of physical encapsulation of APs in solution, in microparticle cores. Such encapsulation conditions are denaturing for certain APs. This is not at all the case as regards the microparticles according to the invention.

In addition, it is particularly surprising and unexpected that the particles based on a poly(AAI)/(polylactide and/or glycolide and/or caprolactone) amphiphilic block copolymer can associate and release APs, in particular proteins, in vivo.

The structure of the HCAP/polyAAI block copolymers and the nature of the AAI amino acids are chosen such that:
the polymer chains spontaneously structure themselves in the form of particles (DPs) that are small in size;
the particles form a colloidal suspension that is stable in water and in physiological medium (pH=6-8);
the DPs associate in the nondissolved colloidal state with proteins or other APs in aqueous medium, via a spontaneous mechanism that does not denature the AP;
the DPs release the APs in physiological medium and, more specifically, in vivo; the kinetics of release depend on the nature of the HCAP/polyAAI copolymer that is the precursor for the DPs.

Thus, by adjusting the specific structure of the copolymer, it is possible to control the phenomena of association and of release of the AP from a kinetic and quantitative point of view.

Preferably, the suspension is characterized in that it is obtained by dissolving the amphiphilic copolymer in an organic solvent and bringing together this solvent and an aqueous liquid.

In order to define the copolymers constituting the particles a little further, it may be indicated that they are of the block type.

Thus, according to a preferred embodiment of the DPs according to the invention:
the AAIs are hydrophilic amino acids AAI;
the HCA/AAI ratio is greater than 0.1;
the absolute length of the HCAP block is greater than 2 monomers, preferably greater than 10 monomers, and more preferably between 20 and 100 monomers.

In the present application, the term “HCA” is intended to mean a constitutive monomer of the HCAP.

Advantageously, the PAA block(s) based on AAIs include at least 5, preferably at least 20, and even more preferably at least 30 to 100, thereof.

Even more preferably, the particles are HCAP/AAI “diblocks”.

The AAI(s) is (are) chosen from amino acids with an ionizable side chain, the natural amino acids Glu and Asp in carboxylic form and/or in the form of salts being particularly preferred.

The PAA blocks constituting particles have, for example, degrees of polymerization dp of between 30 and 600, preferably of between 50 and 200, and even more preferably between 60 and 150.

The present invention is directed not only toward suspensions of naked particles, as defined above, but also particles including at least one active principle AP; preferably, the suspension according to the invention is aqueous and stable. These particles, which may or may not be loaded with AP, are advantageously in a form dispersed in a liquid (suspension), preferably an aqueous liquid, but may also be in the pulverulent solid state, obtained from the DP suspension as defined above.

Hence it ensues that the invention concerns, besides a colloidal suspension (preferably aqueous suspension) of DPs, a pulverulent solid including DPs that is obtained from the suspension according to the invention.

Another essential subject of the invention relates to the preparation of the selected particles (as described above), both in the form of a colloidal suspension and in the form of a pulverulent solid. The method of preparation considered consists essentially in synthesizing precursor HCAP/polyAAI copolymers and in converting them into structured particles.

More specifically, it involves, first of all, a method for preparing the pulverulent solid mentioned above and made up of submicronic particles which can be used in particular for delivering active principle(s) (Aps), these particles being individualized supramolecular arrangements:
based on an amphiphilic copolymer comprising:
   at least one block of α-peptide-linked hydrophilic linear polyamino acid(s) (PAAs), the hydrophilic amino acids AAI constituting this PAA block being identical to or different from one another;
   and at least one block of hydrophobic polymer(s) based on (a) polymer(s) of α-hydroxycarboxylic acid(s) (HCAP)-preferably (a) lactic acid polymer(s) (LAPs) or (a) glycolic acid polymer(s) (GAPs));
capable of forming a colloidal suspension, even in the absence of surfactants;
capable of associating in colloidal suspension in the nondissolved state, with at least one AP and in releasing it, in particular in vivo, in a sustained and/or delayed manner.

This method is characterized in that:
—1)—at least one HCAP (preferably LAP or GAP) block polymer is used or prepared by polymerization of monomers of α-hydroxy-carboxylic acid(s), preferably lactic acid or glycolic acid; this HCAP block being functionalized (advantageously at least one of its ends) with at least one protective reactive group, preferably chosen from the groups comprising BOC-ethanolamine and BOC-aminopropanol (BOC=ButOxyCarbonyl);
—2)—the HCAP polymer block of step —1)—is deprotected;
—3)—a copolymerization of monomers made up of AAI hydrophilic amino acid N-carboxyamino acid anhydrides (NCAs) and/or of AAI hydrophilic amino acid-precursor N-carboxyamino acid anhydrides (NCAs), carrying protective groups, is carried out in the presence of at least one organic solvent, preferably chosen from the group comprising: N-methylpyrrol-idone (NMP), dimethylformamide (DMF), dimethyl sulfoxide (DMSO), dimethylacetamide (DMAc), pyrrolidone and dichloromethane, the latter being more particularly preferred;
—4)—the deprotected HCAP polymer block of step —2)—is added to the polyAAI block polymerization medium, before, during or after the polymerization;
—5)—optionally, the AAI hydrophilic amino acid precursors are deprotected so as to obtain one or more polyAAI blocks;
—6)—the HCAP-polyAAI block copolymer obtained at the end of the preceding steps is precipitated;
—7)—the HCAP-polyAAI block copolymer precipitate obtained in step —6)— is dissolved and this solution is brought together with a liquid containing at least one non-solvent for the HCAP-polyAAI block copolymer, preferably water, this liquid having a pH chosen such that the AAIs of the HCAP-polyAAI block copolymer are at least partly ionized;
—8)—optionally, at least one hydrophilic active principle AP is associated with the particles;
—9)—optionally, the suspension of step —7)— is purified;
—10)—optionally, the suspension of step —7)— is concentrated;
—11)—the liquid medium is eliminated so as to collect the pulverulent solid comprising the particles.

Advantageously, the HCAPs of step —1)— are obtained in a manner known per se, by polymerization of lactide, of glycolide or of caprolactone, or alternatively are commercially available products (polylactide, polylactide/glycolide, polycaprolactone, for example).

Methods for obtaining these HCAPs are described, for example, in the following patents: U.S. Pat. No. 4,835,293, U.S. Pat. No. 5,023,349 and FR 2 692 263.

The deprotection according to step —2)— is carried out in a manner known per se, for example by acid hydrolysis (e.g. trifluoroacetic acid).

The third step of the method is based on the known techniques of N-carboxy(amino acid) anhydride (NCA) polymerization described, for example, in the article “Biopolymers, 15, 1869 (1976)” and in the book by H.R. Kricheldorf “(-Aminoacid-N-carboxy Anhydride and Related Heterocycles” Springer Verlag (1987)”.

Preferably, the functionalized HCAP block(s) is (are) introduced before and/or at the beginning of the polymerization according to step —3)—, which preferably takes place at a temperature of between 20 and 120° C. at normal atmospheric pressure.

Advantageously, this step —3)— is carried out in the presence of at least one cosolvent selected from aprotic solvents (preferably 1,4-dioxane) and/or protic solvents (preferably pyrrolidone) and/or water and/or alcohols, methanol being particularly preferred.

Even more preferably, the NCA-AAIs are NCAs of O-alkylated or O-arylated glutamic acid or aspartic acid, for example NCA-Glu-O-Me, NCA-Glu-O-Et or NCA-Glu-O-Bz (Me=methyl/Et=ethyl/Bz=benzyl).

Other experimental parameters such as:
the concentration of NCA and/or of HCAP block polymer in the organic solvent (preferably dichloromethane);
and/or the concentration or nature of the possible cosolvent, during the synthesis;
the temperature of the reaction mixture;
the mode of addition of the hydrophilic polymer;
the use of reduced pressure;
the reaction time, etc.;
are adjusted to the desired effects that are well known to those skilled in the art.

According to a variant in which the method is interrupted at the end of a step —5a)—which follows on from the end of step —5)—, the HCAP-polyAAI copolymer obtained is precipitated, preferably from water, and this precipitate is collected. This variant corresponds to a batch mode of particle preparation, in which the HCAP-polyAAI copolymer is isolated in the form of a precipitate forming a stable intermediate product. This precipitate may, for example, be filtered, washed and dried.

To perform the association, in step —8)— of one or more APs with the particles, it is possible to use several methods in accordance with the invention.

Nonlimiting examples of these methods are listed below.

According to a first method, the association of APs with the particles is carried out by bringing together a liquid (aqueous or nonaqueous) phase containing the AP and the colloidal suspension of particles.

According to a second method, the association of the AP with the particles is carried out by bringing together an AP in the solid state and the colloidal suspension of the particles. The solid AP may, for example, be in the form of a lyophilisate, a precipitate, a powder, or the like.

According to a third method, the pulverulent solid (polylactide/polyAAI), as described above as product and by virtue of the characteristics for obtaining it, and a liquid (aqueous or nonaqueous) phase containing the AP are brought together.

According to a fourth method, the pulverulent solid, as described above as product and by virtue of the characteristics for obtaining it, and the AP in solid form are brought together. This mixture of solids is then dispersed in a liquid phase, preferably an aqueous solution.

In all these methods, the AP used may be in pure or preformulated form.

In accordance with the optional step —9)—, the impurities (salts) and also the solvent are eliminated by any suitable physical separation treatment, for example by diafiltration (dialysis), filtration, pH modification, chromatography, etc.

This results in a suspension (preferably an aqueous suspension) of structured particles, which can be concentrated [step —10)-], for example by distillation or any other suitable physical means: ultrafiltration, centrifugation.

To separate, in step —11)—, the particles from their liquid suspending medium, the aqueous phase is optionally eliminated, for example by drying (e.g. in an oven), by lyophilization or by any other suitable physical means: ultrafiltration, centrifugation. At the end of this step —11)— a white-colored pulverulent solid is recovered.

It should be noted that the implementation of steps —1)—, —2)—, —3)—, —4)—, —5)—, —6)—, —7)— and, optionally, —8)— of the above method corresponds to a preparation of a colloidal suspension of submicronic particles having a high degree of loading with AP.

During this preparation of colloidal suspension, the polylactide and/or polyglycolide and/or polycaprolactone-poly(AAI) amphiphilic copolymers of step —6)— are placed in an aqueous medium in which at least some of the HCAPs are soluble and at least some of the AANOs are insoluble. The HCAP/polyAAI copolymers exist in the form of nanoparticles in this aqueous medium.

An alternative for preparing the DP suspension according to the invention consists in bringing together the pulverulent solid, as described above as product and by virtue of the method for obtaining it, and an aqueous medium which is not a solvent for the AAIs.

Given the nanometric size of the particles, the suspension can be filtered through sterilizing filters, which makes it possible to readily and less expensively obtain sterile injectable medicinal liquids. The fact that, by virtue of the invention, it is possible to subject the suspension of particles to sterilizing filtration is a considerable asset.

The present invention is also directed toward novel intermediate products of the method described above, characterized in that they consist of HCAP-polyAAI copolymers that are particle precursors.

According to another of its aspects, the invention relates to a suspension and/or a pulverulent solid, as defined above and/or as obtained by means of the method presented above, this suspension and this solid including at least one hydrophilic active principle preferably chosen from:
vaccines;
proteins and/or peptides, among which those most preferably selected are: hemoglobins, cytochromes, albumins, interferons, cytokines, antigens, antibodies, erythropoietin, insulin, growth hormones, factors VIII and IX, interleukins or mixtures thereof, hematopoiesis-stimulating factors;
polysaccharides, heparin being more particularly selected;
nucleic acids, and preferably RNA or DNA oligonucleotides;
non-peptido-protein molecules belonging to various anticancer chemotherapy classes, and in particular anthracyclins and taxoids;
and mixtures thereof.

Finally, the invention relates to a pharmaceutical, nutritional, plant-care or cosmetic specialty product, characterized in that it comprises a suspension or pulverulent solid loaded with hydrophilic AP and as defined above.

According to another of its subjects, the invention is also directed toward the use of these DPs (in suspension or in solid form) loaded with AP, for producing medicinal products of the systems for controlled release of AP type.

They may, for example, be those which can be administered preferably orally, nasally, vaginally, ocularly, subcutaneously, intravenously, intra-muscularly, intradermally, intraperitoneally, Intra-cerebrally or parenterally.

The cosmetic applications that can be envisioned are, for example, compositions comprising an AP associated with the DPs according to the invention and applicable transdermally.

Finally, the invention relates to a pharmaceutical, nutritional, plant-care or cosmetic specialty product, characterized in that it comprises a suspension and/or pulverulent solid loaded with AP and as defined above.

According to another of its subjects, the invention is also directed toward the use of these DPs (in suspension or in solid form) loaded with AP, for producing medicinal products of the system for controlled release of AP type.

In the case of medicinal products, they may, for example, be those which can be administered preferably orally, nasally, vaginally, ocularly, subcutaneously, intravenously, intramuscularly, intradermally, intraperitoneally, intracerebrally or parenterally.

The cosmetic applications that can be envisioned are, for example, compositions comprising an AP associated with the DPs according to the invention and applicable transdermally.

The plant-care products concerned may, for example, be herbicides, pesticides, insecticides, fungicides, etc.

The following examples will make it possible to understand the invention more fully in its various product/method/application aspects. These examples illustrate the preparation of polylactide/PAAI particles which may or may not be loaded with active principles, and they also present the structural characteristics and the properties of these particles.

### DESCRIPTION OF THE FIGURES

FIG. 1:

Isotherm for adsorption of insulin (9.3 mg/ml) onto the dispersion of nanoparticles of Example 8.

FIG. 2:

Blood insulin and blood glucose profiles in normal pigs after administration of a dose of 0.6 IU/kg of insulin adsorbed onto the particles of Example 7.

### EXAMPLES

The synthesis of the block copolymers is carried out in four main steps:
1. polymerization of the lactide with a protected bifunctional initiator;
2. deprotection of the initiator linked to the polymer;
3. polymerization of the second monomer on the deprotected function of the initiator;
4. deprotection of the protective groups on the second monomer.

### Example 1

### poly(lactic acid)₂₀-block-(glutamic acid)₅₀

1.1 BOC-aminopropyl-poly(lactic acid)₂₀: L-lactide (5 g, 34.70 mmol, Aldrich 16101-127) and distilled toluene (27 ml) are introduced into a dry round-bottomed flask and under nitrogen. Heating is carried out for one hour at 80° C. A mixture of tert-butoxycarbonylaminopropanol (0.58 g, 3.30 mmol, Fluka 381029/1) and of freshly distilled toluene (23 ml) is prepared in a second round-bottomed flask and is cooled to −10° C. After the addition of diethylzinc (1.5 ml, 1.65 mmol, 1.1M in toluene, Aldrich 72560-099) to the BOC-aminopropanol, this reaction is allowed to return to ambient temperature, and it is then added to the lactide monomer in order to initiate the polymerization. The polymerization is stopped by adding 4 ml of acetic acid in solution in toluene (10%). The reaction medium is then concentrated in a rotary evaporator and precipitated from a large excess of methanol. The polymer is recovered by filtration and then dried under vacuum. Yield: 98%. Characterizations: Tg: 30-37° C. ¹H NMR (CDCl₃): δ=1.4 ppm (s, 9H, CCH₃), 1.55 ppm (d, 6H, ³J=7.1 Hz, CHCH₃), 1.85 ppm (q, 2H, ³J=6.3 Hz, CH₂CH₂CH₂), 3.1 ppm (q, 2H, ³J=6.2 Hz, CH₂NH), 4.15 ppm (t, 2H, ³J=6.0 Hz, CH₂O), 4.35 ppm (q, 1H, ³J=6.9 Hz, CH₃CHOH), 4.8 ppm (m, 1H, NH), 5.15 ppm (q, 2H, ³J=7.1 Hz, CHCH₃). ^{—}C NMR (CDCl₃): δ=17 ppm (2C, CHCH₃), 20.7 ppm (1C, CH₃CHOH), 28.7 ppm (3C, CH₃C), 29.5 ppm (CH₂CH₂CH₂), 37.5 ppm (CH₂NH), 63.5 ppm (CH₂O), 67 ppm (CH₃CHOH), 69.5 ppm (2C, CH), 156 ppm (NHC═O), 170 ppm (CHC(O)O).
1.2 aminopropyl-poly(lactic acid)₂₀: Polylactide (4 g, 2.65 mmol) and distilled dichloromethane (45 ml) are introduced, under a stream of nitrogen, into a round-bottomed flask. Trifluoroacetic acid (8 ml, 0.1 mol, Sigma 19H3648) is introduced and the solution is placed at ambient temperature for half an hour, with stirring, until no more CO₂ is given off. The solvents of the reaction medium are evaporated off in a rotary evaporator. 40 ml of dichloromethane are added and the solution is washed twice with 40 ml of NaHCO₃ in aqueous solution (5%) and then twice with 40 ml of distilled water, until the pH of the washing water is neutral. The organic phase is dried over MgSO₄ and then filtered. The solvent is evaporated off in a rotary evaporator, and the polymer is then dried under vacuum. Yield: 95%. Characterizations: ¹H NMR (CDCl₃): δ=1.55 ppm (d, 6H, ³J=7.1 Hz, CH₃), 1.85 ppm (q, 2H, ³J=6.3 Hz, CH₂CH₂CH₂), 2.8 ppm (q, 2H, J=6.2 Hz, CH₂NH₂), 4.15 ppm (t, 2H, ³J=6.0 Hz, CH₂O); 4.35 ppm (q, 1H, ³J=6.9 Hz, CH₃CHOH), 5.15 ppm (q, 2H, ³J=7.1 Hz, CH).
1.3 poly(benzyl glutamate)₅₀-propyl-poly(lactic acid)₂₀: Benzyl L-glutamate N-carboxyanhydride (8.68 g, 33.0 mmol) is introduced into a round-bottomed flask. The deprotected polylactide (1 g, 0.66 mmol) is solubilized in freshly distilled dichloromethane (40 ml) and then introduced by means of a hollow tube. The reaction medium is placed at ambient temperature for three hours with magnetic stirring. The solvent is evaporated off in a rotary evaporator and the polymer is then dried under primary vacuum. Yield: 85%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH), 5.25 ppm (m, 2H, CH₂Ph), 7.10 ppm (m, 5H, Ph). ¹³C NMR (DMSO d6): δ=17 ppm (CH₃), 27 ppm (CH₂CH₂COO), 30 ppm (CH₂CH₂COO), 52 ppm (O═CCHNH), 66 ppm (CH₂Ph), 128-136 ppm (Ph), 168-170 ppm (OC═O), 172 ppm (NC═O).
1.4 poly(glutamic acid) so-propyl-poly(lactic acid)₂₀: The copolymer (5 g, 20 mmol of benzyl ester) is introduced into a round-bottomed flask and solubilized in trifluoroacetic acid (44 ml, 0.57 mol) at 10° C. Methanesulfonic acid (44 ml, 0.68 mol) and anisole (11 ml, 0.10 mol) are introduced under a stream of nitrogen and the reaction medium is left for three hours with stirring. The polymer is precipitated from a large excess of cold ethyl ether, recovered by filtration, washed with ethyl ether and dried under vacuum. Yield: 99%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH). ¹³C NMR (DMSO d6): δ=17 ppm (CH₃), 27 ppm (CH₂CH₂COOH), 30 ppm (CH₂CH₂COOH), 52 ppm (OCCHNH), 69 ppm (CH), 168-170 ppm (O═CO), 172 ppm (O═CNH).

### Example 2

### poly(lactic acid)₃₀-block-(glutamic acid)₈₀

2.1 BOC-aminopropyl-poly(lactic acid)₃₀: The L-lactide (5 g, 34.70 mmol, Aldrich 16101-127) is introduced, in a glove box, under a nitrogen atmosphere, into a pre-flamed round-bottomed flask. The freshly distilled toluene (27 ml) is introduced, by means of a hollow tube, into the round-bottomed flask, which is placed at 80° C. for one hour with magnetic stirring. The tert-butoxy-carbonylaminopropanol (0.40 g, 2.30 mmol, Fluka 381029/1) and the freshly distilled toluene (23 ml) are introduced into a pre-flamed round-bottomed flask and placed in a bath at −10° C. with magnetic stirring. The solution of diethylzinc in toluene (1.0 ml, 1.15 mmol, 1.1M, Aldrich 72560-099) is introduced dropwise into this solution. The reaction medium is then left at ambient temperature with magnetic stirring. After one hour, the solution of L-lactide is introduced, under a stream of nitrogen, into the reaction medium, which is then placed at 80° C. for one hour with stirring. The polymerization is stopped by adding 4 ml of acetic acid in solution in toluene (10%). The reaction medium is then concentrated in a rotary evaporator and precipitated from a large excess of cold methanol. The precipitated polymer is recovered by filtration and then dried under primary vacuum. Yield: 98%. Characterizations: Tg: 30-37° C. ¹H NMR (CDCl₃): δ=1.4 ppm (s, 9H, CCH₃), 1.55 ppm (d, 6H, ³J=7.1 Hz, CHCH₃), 1.85 ppm (q, 2H, ³J=6.3 Hz, CH₂CH₂CH₂), 3.1 ppm (q, 2H, ³J=6.2 Hz, CH₂NH), 4.15 ppm (t, 2H, ³J=6.0 Hz, CH₂O), 4.35 ppm (q, 1H, ³J=6.9 Hz, CH₃CHOH), 4.8 ppm (m, 1H, NH), 5.15 ppm (q, 2H, ³J=7.1 Hz, CHCH₃). ¹³C NMR (CDCl₃): δ=17 ppm (2C, CHCH₃), 20.7 ppm (1C, CH₃CHOH), 28.7 ppm (3C, CH₃C), 29.5 ppm (CH₂CH₂CH₂), 37.5 ppm (CH₂NH), 63.5 ppm (CH₂O), 67 ppm (CH₃CHOH), 69.5 ppm (2C, CH), 156 ppm (NHC═O), 170 ppm (CHC(O)O).
2.2 aminopropyl-poly(lactic acid)₃₀: The polylactide (4 g, 1.85 mmol) and the freshly distilled dichloromethane (45 ml) are introduced, under a stream of nitrogen, into a pre-flamed round-bottomed flask connected to a bubbling device. Trifluoroacetic acid (8 ml, 0.1 mol, Sigma 19H3648) is introduced and the solution is placed at ambient temperature for half an hour, with stirring, until no more CO₂ is given off. The solvents of the reaction medium are evaporated off in a rotary evaporator. The polylactide is solubilized in 40 ml of dichloromethane. This organic phase is washed twice with 40 ml of NaHCO₃ in aqueous solution (5%) and then twice with 40 ml of distilled water until the pH of the washing water is neutral. The organic phase is then dried over MgSO₄ and then filtered. The solvent is evaporated off in a rotary evaporator and the polymer is then dried under primary vacuum. Yield: 95%. Characterizations: ¹H NMR (CDCl₃): δ=1.55 ppm (d, 6H, ³J=7.1 Hz, CH₃), 1.85 ppm (q, 2H, ³J=6.3 Hz, CH₂CH₂CH₂), 2.8 ppm (q, 2H, ³J=6.2 Hz, CH₂NH₂), 4.15 ppm (t, 2H, ³J=6.0 Hz, CH₂O), 4.35 ppm (q, 1H, ³J=6.9 Hz, CH₃CHOH), 5.15 ppm (q, 2H, ³J=7.1 Hz, CH).
2.3 poly(benzyl glutamate) so-propyl-poly(lactic acid)₃₀: Benzyl L-glutamate N-carboxyanhydride (9.74 g, 37.0 mmol) provided by Flamel Technologies is weighed out and introduced, in a glove box, under a nitrogen atmosphere, into a pre-flamed round-bottomed flask. The deprotected polylactide (1 g, 0.46 mmol) is solubilized in freshly distilled dichloromethane (45 ml) and then introduced by means of a hollow tube. The reaction medium is placed at ambient temperature for three hours with magnetic stirring. The solvent is evaporated off in a rotary evaporator and the polymer is then dried under primary vacuum. Yield: 85%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH), 5.25 ppm (m, 2H, CH₂Ph), 7.10 ppm (m, 5H, Ph). ¹³C NMR (DMSO d6): δ=17 ppm (CH₃), 27 ppm (CH₂CH₂COO), 30 ppm (CH₂CH₂COO), 52 ppm (O═CCHNH), 66 ppm (CH₂Ph), 128-136 ppm (Ph), 168-170 ppm (OC═O), 172 ppm (NC═C).
2.4 poly(glutamic acid)₈₀-propyl-poly(lactic acid)₃₀: The copolymer (5 g, 20.3 mmol of benzyl ester) is introduced, under a stream of nitrogen, into a pre-flamed round-bottomed flask. It is solubilized in trifluoroacetic acid (44 ml, 0.57 mol). The solution is then placed at 10° C., with stirring. Methanesulfonic acid (44 ml, 0.68 mol) and anisole (11 ml, 0.10 mol) are introduced under a stream of nitrogen. The reaction medium is left at 10° C. for three hours with stirring, and is then precipitated from a large excess of cold ethyl ether. The precipitated polymer is recovered by filtration, washed with ethyl ether and dried under primary vacuum. Yield: 99%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH). ¹³C NMR (DMSO d6): δ=17 ppm (CH₃), 27 ppm (CH₂CH₂COOH), 30 ppm (CH₂CH₂COOH), 52 ppm (OCCHNH), 69 ppm (CH), 168-170 ppm (O═CO), 172 ppm (O═CNH).

### Example 3

### poly(lactic acid)₅₀-block-(glutamic acid)₅₀

3.1 BOC-aminopropyl-poly(lactic acid)₅₀: The L-lactide (5 g, 34.70 mmol, Aldrich 16101-127) is weighed out and introduced, in a glove box, under a nitrogen atmosphere, into a pre-flamed round-bottomed flask. The freshly distilled toluene (27 ml) is introduced, by means of a hollow tube, into the round-bottomed flask, which is placed at 80° C. for one hour with magnetic stirring. The tert-butoxycarbonylaminopropanol (0.24 g, 1.3 mmol, Fluka 381029/1) and the freshly distilled toluene (23 ml) are introduced into a pre-flamed round-bottomed flask placed in a bath at −10° C. with magnetic stirring. The solution of diethylzinc in toluene (0.63 ml, 0.69 mmol, 1.1M, Aldrich 72560-099) is introduced dropwise into this solution. The reaction medium is then left at ambient temperature with magnetic stirring. After one hour, the solution of L-lactide is introduced, under a stream of nitrogen, into the reaction medium, which is then placed at 80° C. for one hour with stirring. The polymerization is stopped by adding 4 ml of acetic acid in solution in toluene (10%). The reaction medium is then concentrated in a rotary evaporator and precipitated from a large excess of cold methanol. The precipitated polymer is recovered by filtration and then dried under primary vacuum. Yield: 98%. Characterizations: Tg: 30-37° C. ¹H NMR (CDCl₃): δ=1.4 ppm (s, 9H, CCH₃), 1.55 ppm (d, 6H, ³J=7.1 Hz, CHCH₃), 1.85 ppm (q, 2H, ³J=6.3 Hz, CH₂CH₂CH₂), 3.1 ppm (q, 2H, ³J=6.2 Hz, CH₂NH), 4.15 ppm (t, 2H, ³J=6.0 Hz, CH₂O), 4.35 ppm (q, 1H, ³J=6.9 Hz, CH₃CHOH), 4.8 ppm (m, 1H, NH), 5.15 ppm (q, 2H, ³J=7.1 Hz, CHCH₃). ¹³C NMR (CDCl₃): δ=17 ppm (2C, CHCH₃), 20.7 ppm (1C, CH₃CHOH), 28.7 ppm (3C, CH₃C), 29.5 ppm (CH₂CH₂CH₂), 37.5 ppm (CH₂NH₂), 63.5 ppm (CH₂O), 67 ppm (CH₃CHOH), 69.5 ppm (2C, CH), 156 ppm (NHC═O), 170 ppm (CHC(O)O).
3.2 aminopropyl-poly(lactic acid)₅₀: The polylactide (4 g, 1.11 mmol) and the freshly distilled dichloromethane (45 ml) are introduced, under a stream of nitrogen, into a pre-flamed round-bottomed flask connected to a bubbling device. Trifluoroacetic acid (80 ml, 0.1 mol, Sigma 19H3648) is introduced and the solution is placed at ambient temperature for half an hour, with stirring, until no more CO₂ is given off. The solvents of the reaction medium are evaporated off in a rotary evaporator. The polylactide is solubilized in 40 ml of dichloromethane. This organic phase is washed twice with 40 ml of NaHCO₃ in aqueous solution (5%) and then twice with 40 ml of distilled water until the pH of the washing water is neutral. The organic phase is then dried over MgSO₄ and then filtered. The solvent is evaporated off in a rotary evaporator and the polymer is then dried under primary vacuum. Yield: 95%. Characterizations: ¹H NMR (CDCl₃): δ=1.55 ppm (d, 6H, ³J=7.1 Hz, CH₃), 1.85 ppm (q, 2H, ³J=6.3 Hz, CH₂CH₂CH₂), 2.8 ppm (q, 2H, ³J=6.2 Hz, CH₂NH₂), 4.15 ppm (t, 2H, ³J=6.0 Hz, CH₂O), 4.35 ppm (q, 1H, ³J=6.9 Hz, CH₃CHOH), 5.15 ppm (q, 2H, ³J=7.1 Hz, CH).
3.3 poly(benzyl glutamate)₅₀-propyl-poly(lactic acid)₅₀: Benzyl L-glutamate N-carboxyanhydride (3.65 g, 13.8 mmol) is weighed out and introduced, in a glove box, under a nitrogen atmosphere, into a pre-flamed round-bottomed flask. The deprotected polylactide (1 g, 0.27 mmol) is solubilized in freshly distilled dichloromethane (17 ml) and then introduced by means of a hollow tube. The reaction medium is placed at ambient temperature for three hours with magnetic stirring. The solvent is evaporated off in a rotary evaporator and the polymer is then dried under primary vacuum. Yield: 85%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH), 5.25 ppm (m, 2H, CH₂Ph), 7.10 ppm (m, 5H, Ph). ¹³C NMR (DMSO d6): δ=17 ppm (CH₃), 27 ppm (CH₂CH₂COO), 30 ppm (CH₂CH₂COO), 52 ppm (O═CCHNH), 66 ppm (CH₂Ph), 128-136 ppm (Ph), 168-170 ppm (OC═O), 172 ppm (NC═O).
3.4 poly(glutamic acid)₅₀-propyl-poly(lactic acid) 50: The copolymer (3 g, 10.27 mmol of benzyl ester) is introduced, under a stream of nitrogen, into a pre-flamed round-bottomed flask. It is solubilized in trifluoroacetic acid (22.5 ml, 0.29 mol). The solution is then placed at 10° C., with stirring. Methanesulfonic acid (22.5 ml, 0.35 mol) and anisole (5.5 ml, 0.05 mol) are introduced under a stream of nitrogen. The reaction medium is left at 10° C. for 3 hours with stirring, and is then precipitated from a large excess of cold ethyl ether. The precipitated polymer is recovered by filtration, washed with ethyl ether and dried under primary vacuum. Yield: 99%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂CH₂C═O), 4.60 ppm (m, 1H, O═CCHNH), 5.0 ppm (m, 2H, CH). ¹³C NMR (DMSO d6): δ=17 ppm (CH₃), 27 ppm (CH₂CH₂COOH), 30 ppm (CH₂CH₂COOH), 52 ppm (OCCHNH), 69 ppm (CH), 168-170 ppm (O═CO), 172 ppm (O═CNH).

### Example 4

### poly(lactic acid)₈₀-block-(glutamic acid)₂₀

4.1 BOC-aminopropyl-poly(lactic acid)₈₀: L-lactide (5.2 g, 36.09 mmol, Aldrich 16101-127) and distilled toluene (27 ml) are introduced into a dry round-bottomed flask and under nitrogen. Heating is carried out for one hour at 80° C. A mixture of tert-butoxycarbonylaminopropanol (0.16 g, 0.91 mmol, Fluka 381029/1) and of freshly distilled toluene (23 ml) is prepared in a second round-bottomed flask and is cooled to −10° C. After the addition of diethylzinc (0.4 ml, 0.44 mmol, 1.1M in toluene, Aldrich 72560-099) to the BOC-aminopropanol, this reaction is allowed to return to ambient temperature, and it is then added to the lactide monomer in order to initiate the polymerization. The polymerization is stopped by adding 0.5 ml of acetic acid in solution in toluene (10%). The reaction medium is then concentrated in a rotary evaporator and precipitated from a large excess of methanol. The polymer is recovered by filtration and then dried under vacuum. Yield: 98%. Characterizations: Tg: 30-37° C. ¹H NMR (CDCl₃): δ=1.4 ppm (s, 9H, CCH₃), 1.55 ppm (d, 6H, ³J=7.1 Hz, CHCH₃), 1.85 ppm (q, 2H, ³J=6.3 Hz, CH₂CH₂CH₂), 3.1 ppm (q, 2H, ³J=6.2 Hz, CH₂NH), 4.15 ppm (t, 2H, ³J=6.0 Hz, CH₂O), 4.35 ppm (q, 1H, ³J=6.9 Hz, CH₃CHOH), 4.8 ppm (m, 1H, NH), 5.15 ppm (q, 2H, ³J=7.1 Hz, CHCH₃). ¹³C NMR (CDCl₃): δ=17 ppm (2C, CHCH₃), 20.7 ppm (1C, CH₃CHOH), 28.7 ppm (3C, CH₃C), 29.5 ppm (CH₂CH₂CH₂), 37.5 ppm (CH₂NH), 63.5 ppm (CH₂O), 67 ppm (CH₃CHOH), 69.5 ppm (2C, CH), 156 ppm (NHC═O), 170 ppm (CHC(O)O).
4.2 aminopropyl-poly(lactic acid)₈₀: Polylactide (4.5 g, 2.98 mmol) and distilled dichloromethane (54 ml) are introduced, under a stream of nitrogen, into a round-bottomed flask. Trifluoro-acetic acid (9 ml, 0.11 mol, Sigma 19H3648) is introduced and the solution is placed at ambient temperature for half an hour, with stirring, until no more CO₂ is given off. The solvents of the reaction medium are evaporated off in a rotary evaporator. 50 ml of dichloromethane are added and the solution is washed twice with 50 ml of NaHCO₃ in aqueous solution (5%) and then twice with 50 ml of distilled water until the pH of the washing water is neutral. The organic phase is dried over MgSO₄ and then filtered. The solvent is evaporated off in a rotary evaporator, and the polymer is then dried under vacuum. Yield: 95%. Characterizations: ¹H NMR (CDCl₃): δ=1.55 ppm (d, 6H, ³J=7.1 Hz, CH₃), 1.85 ppm (q, 2H, ³J=6.3 Hz, CH₂CH₂CH₂), 2.8 ppm (q, 2H, ³J=6.2 Hz, CH₂NH₂), 4.15 ppm (t, 2H, ³J=6.0 Hz, CH₂O), 4.35 ppm (q, 1H, ³J=6.9 Hz, CH₃CHOH), 5.15 ppm (q, 2H, ³J=7.1 Hz, CH).
4.3 poly(benzyl glutamate) 20-propyl-poly(lactic acid)₈₀: Benzyl L-glutamate N-carboxyanhydride (2.70 g, 10.26 mmol) is introduced into a round-bottomed flask. The deprotected polylactide (3 g, 1.98 mmol) is solubilized in freshly distilled dichloromethane (15 ml) and then introduced by means of a hollow tube. The reaction medium is placed at ambient temperature for three hours with magnetic stirring. The solvent is evaporated off in a rotary evaporator and the polymer is then dried under primary vacuum. Yield: 85%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH), 5.25 ppm (m, 2H, CH₂Ph), 7.10 ppm (m, 5H, Ph) ¹³C NMR (DMSO d6): δ=17 ppm (CH₃), 27 ppm (CH₂CH₂COO), 30 ppm (CH₂CH₂COO), 52 ppm (O═CCHNH), 66 ppm (CH₂Ph), 128-136 ppm (Ph), 168-170 ppm (OC═O), 172 ppm (NC═O).
4.4 poly(glutamic acid) 20-propyl-poly(lactic acid)₈₀: The copolymer (5.2 g, 20.8 mmol of benzyl ester) is introduced into a round-bottomed flask and solubilized in trifluoroacetic acid (22 ml, 0.29 mol) at 10° C.. Methanesulfonic acid (22 ml, 0.34 mol) and anisole (5.6 ml, 0.05 mol) are introduced under a stream of nitrogen and the reaction medium is left at 10° C. for three hours with stirring. The polymer is precipitated from a large excess of cold ethyl ether, recovered by filtration, washed with ethyl ether and dried under vacuum. Yield: 99%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH). ¹³C NMR (DMSO d6): δ=17 ppm (CH₃), 27 ppm (CH₂CH₂COOH), 30 ppm (CH₂CH₂COOH), 52 ppm (OCCHNH) 69 ppm (CH), 168-170 ppm (O═CO), 172 ppm (O═CNH).

### Example 5

### poly(lactic acid)₃₀-block-(glutamic acid) 100

5.1 BOC-aminopropyl-poly(lactic acid)₃₀: The L-lactide (6 g, 41.64 mmol, Aldrich 16101-127) is introduced, in a glove box, under a nitrogen atmosphere, into a pre-flamed round-bottomed flask. The freshly distilled toluene (27 ml) is introduced by means of a hollow tube into the round-bottomed flask, which is placed at 80° C. for one hour with magnetic stirring. The tert-butoxycarbonylaminopropanol (0.73 g, 4.20 mmol), Fluka 381029/1) and the freshly distilled toluene (23 ml) are introduced into a pre-flamed round-bottomed flask and placed in a bath at −10° C. with magnetic stirring. The solution of diethylzinc in toluene (1.9 ml, 2.19 mmol, 1.1M, Aldrich 72560-099) is introduced dropwise into this solution. The reaction medium is then left at ambient temperature with magnetic stirring. After one hour, the solution of L-lactide is introduced, under a stream of nitrogen, into the reaction medium, which is then placed at 80° C. for one hour with stirring. The polymerization is stopped by adding 5 ml of acetic acid in solution in toluene (10%). The reaction medium is then concentrated in a rotary evaporator and precipitated from a large excess of cold methanol. The precipitated polymer is recovered by filtration and then dried under primary vacuum. Yield: 98%. Characterizations: Tg: 30-37° C. ¹H NMR (CDCl₃): δ=1.4 ppm (s, 9H, CCH₃), 1.55 ppm (d, 6H, ³J=7.1 Hz, CHCH₃), 1.85 ppm (q, 2H, ³J=6.3 Hz, CH₂CH₂CH₂), 3.1 ppm (q, 2H, ³J=6.2 Hz, CH₂NH), 4.15 ppm (t, 2H, ³J=6.0 Hz, CH₂O), 4.35 ppm (q, 1H, ³J=6.9 Hz, CH₃CHOH), 4.8 ppm (m, 1H, NH), 5.15 ppm (q, 2H, ³J=7.1 Hz, CHCH₃). ¹³C NMR (CDCl₃): δ=17 ppm (2C, CHCH₃), 20.7 ppm (1C, CH₃CHOH), 28.7 ppm (3C, CH₃C), 29.5 ppm (CH₂CH₂CH₂), 37.5 ppm (CH₂NH), 63.5 ppm (CH₂O), 67 ppm (CH₃CHOH), 69.5 ppm (2C, CH), 156 ppm (NHC═O), 170 ppm (CHC(O)O).
5.2 aminopropyl-poly(lactic acid)₃₀: The polylactide (3 g, 1.39 mmol) and the freshly distilled dichloromethane (36 ml) are introduced, under a stream of nitrogen, into a pre-flamed round-bottomed flask connected to a bubbling device. Trifluoroacetic acid (6 ml, 0.08 mol, Sigma 19H3648) is introduced and the solution is placed at ambient temperature for half an hour, with stirring, until no more CO₂ is given off. The solvents of the reaction medium are evaporated off in a rotary evaporator. The polylactide is solubilized in 40 ml of dichloromethane. This organic phase is washed twice with 40 ml of NaHCO₃ in aqueous solution (5%) and then twice with 40 ml of distilled water until the pH of the washing water is neutral. The organic phase is then dried over MgSO₄ and then filtered. The solvent is evaporated off in a rotary evaporator and the polymer is then dried under primary vacuum. Yield: 97%. Characterizations: ¹H NMR (CDCl₃): δ=1.55 ppm (d, 6H, ³J=7.1 Hz, CH₃), 1.85 ppm (q, 2H, ³J=6.3 Hz, CH₂CH₂CH₂), 2.8 ppm (q, 2H, ³J=6.2 Hz, CH₂NH₂), 4.15 ppm (t, 2H, ³J=6.0 Hz, CH₂O), 4.35 ppm (q, 1H, ³J=6.9 Hz, CH₃CHOH), 5.15 ppm (q, 2H, ³J=7.1 Hz, CH).
5.3 poly(benzyl glutamate)₁₀₀-propyl-poly(lactic acid)₃₀: Benzyl glutamate N-carboxyanhydride (33 g, 125.4 mmol) provided by Flamel Technologies is weighed out and introduced, in a glove box, under a nitrogen atmosphere, into a pre-flamed round-bottomed flask. The deprotected polylactide (2.9 g, 1.33 mmol) is solubilized in freshly distilled dichloromethane (165 ml) and then introduced by means of a hollow tube. The reaction medium is placed at ambient temperature for three hours with magnetic stirring. The solvent is evaporated off in a rotary evaporator and the polymer is then dried under primary vacuum. Yield: 93%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH), 5.25 ppm (m, 2H, CH₂Ph), 7.10 ppm (m, 5H, Ph). ¹³C NMR (DMSO d6): δ=17 ppm (CH₃), 27 ppm (CH₂CH₂COO), 30 ppm (CH₂CH₂COO), 52 ppm (O═CCHNH), 66 ppm (CH₂Ph), 128-136 ppm (Ph), 168-170 ppm (OC═O), 172 ppm (NC═O).
5.4 poly(glutamic acid)₁₀₀-propyl-poly(lactic acid)₃₀: The copolymer (11 g, 44.66 mmol of benzyl ester) is introduced, under a stream of nitrogen, into a pre-flamed round-bottomed flask. It is solubilized in trifluoroacetic acid (100 ml, 1.30 mol). The solution is then placed at 10° C., with stirring. Methanesulfonic acid (100 ml, 1.55 mol) and anisole (25 ml, 0.23 mol) are introduced under a stream of nitrogen. The reaction medium is left at 10° C. for three hours with stirring, and is then precipitated from a large excess of cold ethyl ether. The precipitated polymer is recovered by filtration, washed with ethyl ether and dried under primary vacuum. Yield: 99%. Characterizations: ¹H NMR (TFA d): δ=1.55 ppm (m, 6H, CH₃), 1.95 ppm (m, 2H, CH₂CH₂C═O), 2.35 ppm (m, 2H, CH₂CH₂C═O), 4.60 ppm (m, 1H, O═CCHNH), 5.0 ppm (m, 2H, CH). ¹³C NMR (DMSO d6): δ=17 ppm (CH₃), 27 ppm (CH₂CH₂COOH), 30 ppm (CH₂CH₂COOH), 52 ppm (OCCHNH), 69 ppm (CH), 168-170 ppm (O═CO), 172 ppm (O═CNH).

### Example 6

### Formation of Nanoparticles from the Polymer of Example 4

0.5 g of powder of the polymer of Example 4 is dissolved in 20 g of a 90/10 w/w THF/ethanol mixture. The solution is poured dropwise into 4 volumes of a 0.1M aqueous phosphate buffer solution. The dispersion obtained is a diffusing dispersion.

### Example 7

### Measurement of the Hydrodynamic Diameter of the Nanoparticles of Example 6

The diffusing dispersion of Example 6 is diafiltered through a Biomax YM 300 membrane. The nanoparticles are concentrated in the retentate. They are then dialyzed at constant volume against 800 ml of weakly buffered water (2×10⁻⁴ M phosphate buffer without salt). The hydrodynamic diameter of the particles, determined by dynamic light scattering, is 240 nm.

### Example 8

### Measurement of the Maximum Amount of Insulin Absorbed onto the Polymer Particles of Example 7

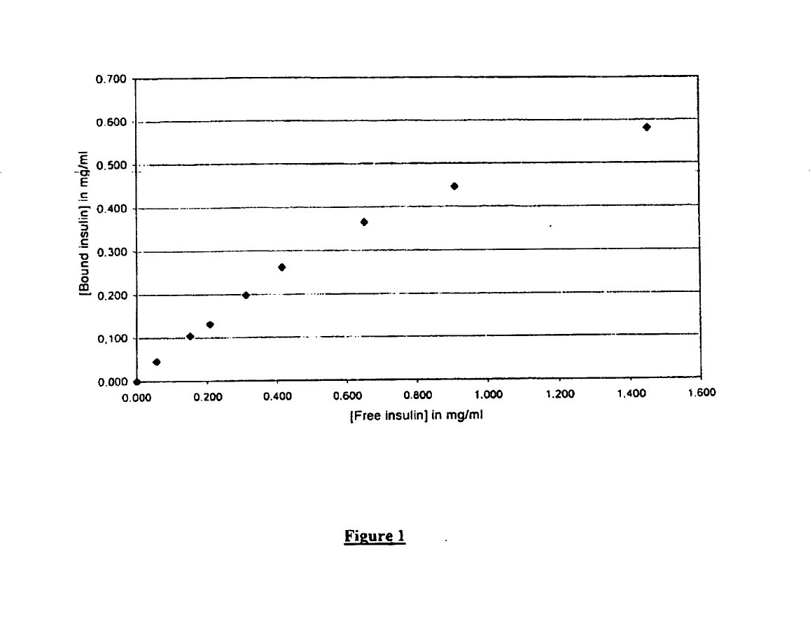

The nanoparticles of Example 7, concentrated to 10 mg/ml under isotonic conditions at pH 7.4, are brought into contact, at 25° C. for 16 hours, with increasing concentrations of human recombinant insulin in solution. The amount of free insulin, i.e. insulin not adsorbed onto the nanoparticles, is determined by steric exclusion chromatography. For this purpose, the preparation is injected into a Toso Haas TSK G4000 PWXL column. The free insulin is detected by means of an Agilent Series 1100 UV-detector at 214 nm. The adsorption isotherm of FIG. 1 is thus obtained. The value at the plateau of this isotherm makes it possible to determine the maximum amount, Am of insulin adsorbed per unit of mass of the dry copolymer. Am=6% w/w is found.

### Example 9

### Formation of Nanoparticles from the Polymer of Example 5

The polymer of Example 5 is obtained in concentrated ethanolic solution at 26.7 g/l. This solution is directly poured dropwise into 4 volumes of a 0.1 M aqueous phosphate buffer solution. The dispersion obtained is a diffusing dispersion.

### Example 10

### Measurement of the Hydrodynamic Diameter of the Nanoparticles of Example 9

The diffusing dispersion of Example 9 is diafiltered through a Biomax YM 300 membrane. The nanoparticles are found to be concentrated in the retentate. They are then dialyzed at constant volume against 800 ml of weakly buffered water (2×10⁻⁴ M phosphate buffer without salt). The hydrodynamic diameter of the particles, determined by dynamic light scattering, is 220 nm.

### Example 11

### Measurement of the Maximum Amount of Insulin Absorbed onto the Particles of Example 10

By carrying out a procedure identical to Example 8, the isotherm for adsorption for insulin onto the nanoparticles of the polymer of Example 5 is obtained. The value at the plateau of this isotherm makes it possible to determined the maximum amount, Am, of the insulin adsorbed per unit of mass of the dry copolymer. Am=1% w/w is found.

### Example 12

### Characterization of the Nanoparticles of the Polymer of Example 3

The nanoparticles of the copolymer of Example 3 are prepared and isolated according to the method disclosed in Examples 6 and 9. The hydrodynamic diameter of the nanoparticles, measured by dynamic light scattering, is 450 nm. The maximum amount of insulin adsorbed onto these nanoparticles is measured as disclosed in Examples 8 and 11. Am=2.5% w/w is found.

### Example 13

### Formation of Nanoparticles from the Polymer of Example 3

500 mg of polymer according to Example 3 are dissolved in 100 ml of DMF in 10 minutes at 60° C. This solution is poured slowly into a volume of 200 ml of diisopropyl ether at −40° C., vigorously stirred. The solution is left to stand at ambient temperature for 2 hours and then centrifuged at 1500 rpm for 20 min. The pellet is filtered through a Büchner No. 4 funnel and the precipitate is washed with diisopropyl ether. The precipitate is dried under vacuum from a vane pump.

### Example 14

### Pharmacokinetics and Pharmacodynamics of the DPs Loaded with Insulin in Normal Fasting Dogs

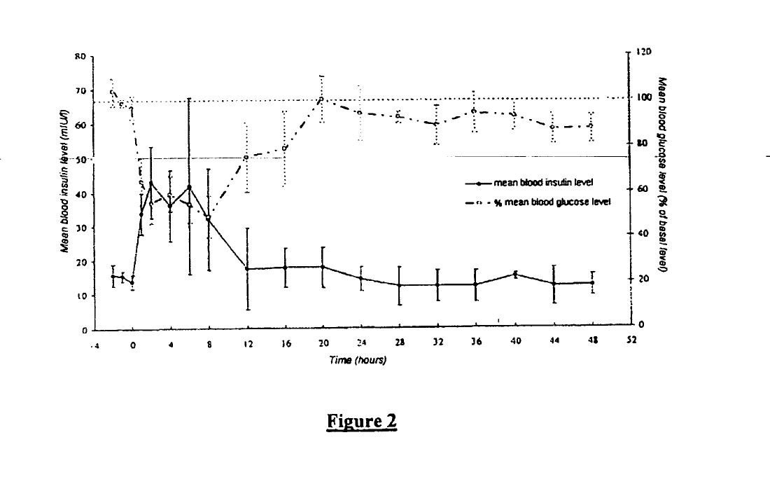

The preparation of HCAP-polyAAI microparticles of Example 7 associated with insulin (Basulin®) of Example 8 was injected into dogs that had been made diabetic by total pancreatectomy, and had been fasting since the previous evening. The administration of the preparation, at 11 o'clock in the morning by the thoracic subcutaneous route, was carried out at the dose of 0.5 IU/kg of insulin per kg of live weight of the animal. The volume administered is between 0.18 and 0.24 ml. At time −4, −2, 0, 1, 2, 4, 6, 8, 12, 16, 20, 24, 28, 32, 36, 40, 44 and 48 hours, 1 ml of blood was taken by puncture of the jugular vein, under vacuum on a sodium heparinate tube. 30 μl of total blood are used extemporaneously to measure the blood glucose level. The tube is then centrifuged and allowed to settle, and the plasma is stored at −20° C. for assaying of the insulin. The results given in FIG. 2 hereinafter show insulin release up to 12 hours (solid line) and a considerable blood glucose-lowering effect which is sustained up to 20 hours (discontinuous line) after the injection.

This example demonstrates the non-denaturation of the insulin in the presence of DPs according to the invention.

In addition, this example shows that the nanoparticles according to the invention are made up of DPs which can be used effectively for the modified release of proteins.

1.-19. (canceled)

20. A suspension of submicronic particles that are stable in the absence of surfactants, wherein the particles are capable of associating in the suspension in a nondissolved state with at least one active principle such that the active principle is released in a sustained and/or delayed manner in vivo;
wherein the suspension can be obtained spontaneously in the absence of surfactant by reacting at least one amphiphilic copolymer with a liquid that is not a solvent for hydrophilic amino acids, and
wherein the particles being individualized supramolecular arrangements based on an amphiphilic copolymer comprising:
at least one block of α-peptide-linked hydrophilic linear polyamino acid, wherein the hydrophilic amino acids are in an at least partially ionized form; and
at least one block of at least one hydrophobic polymer comprising at least one α-hydroxycarboxylic acid polymer.

21. The suspension according to claim 20, wherein the at least one α-hydroxycarboxylic acid polymer is selected from the group consisting of: lactic acid polymer, glycolic acid polymer and a mix thereof.

22. The suspension according to claim 20, wherein the at least one amphiphilic copolymer is first dissolved in an organic solvent before the addition of the liquid.

23. The suspension according to claim 20, wherein the ratio of α-hydroxycarboxylic acid polymer to hydrophilic amino acids is greater than 0.1, and the absolute length of the α-hydroxycarboxylic acid polymer is greater than 2 monomers.

24. The suspension according to claim 23, wherein the absolute length of the α-hydroxycarboxylic acid polymer is greater than 10 monomers.

25. The suspension according to claim 23, wherein the absolute length of the α-hydroxycarboxylic acid polymer is between about 20 and 60 monomers.

26. The suspension according to claim 20, wherein the α-peptide-linked hydrophilic linear polyamino acid blocks include at least 5 hydrophilic amino acids.

27. The suspension according to claim 20, wherein the α-peptide-linked hydrophilic linear polyamino acid blocks include at least 20 hydrophilic amino acids.

28. The suspension according to claim 20, wherein the α-peptide-linked hydrophilic linear polyamino acid blocks include between 30 and 100 hydrophilic amino acids.

29. The suspension according to claim 20, wherein the at least one block of α-peptide-linked hydrophilic linear polyamino acid and the at least one block of hydrophobic polymer are diblocks.

30. The suspension according to claim 20, wherein the hydrophilic amino acids are selected from the group comprising: amino acids with an ionizable side chain, glutamate in carboxylic form, glutamate in a salt form, aspartate in carboxylic form, aspartate in a salt form and a mix thereof.

31. The suspension according to claim 20, wherein the suspension is an aqueous solution.

32. The suspension according to claim 20, wherein the suspension comprises a pulverulent solid.

33. A pharmaceutical, nutritional, plant-care or cosmetic specialty product that comprises the suspension of claim 20.

34. The suspension according to claim 20, wherein the suspension comprises at least one hydrophilic active principle.

35. The suspension according to claim 34, wherein the at least one hydrophilic active principle is selected from the group comprising: vaccines, proteins, peptides, hemoglobins, cytochromes, albumins, interferons, cytokines, antigens, antibodies, erythropoietin, insulin, growth hormones, factors VIII and IX, interleukins, hematopoiesis-stimulating factors, polysaccharides, heparin, nucleic acids, anti-cancer non-peptido-protein molecules, anthracyclins, taxoids, and mixtures thereof.

36. A pulverulent solid obtained from a suspension according to claim 20.

37. A method of preparing a suspension, wherein the method comprises:
(i) at least one α-hydroxycarboxylic acid polymer prepared by polymerization of α-hydroxycarboxylic acid monomers and comprising at least one protected reactive group, wherein the at least one α-hydroxycarboxylic acid polymer is deprotected;
(ii) at least partially ionizable hydrophilic amino acid that is copolymerized in the presence of at least one organic solvent; and
(iii) the at least one deprotected α-hydroxycarboxylic acid polymer block of step (i) is added to the poly amino acid block polymerization medium of step (ii) before, during or after the step (ii) polymerization to form a block copolymer.

38. The method according to claim 37, wherein the α-hydroxycarboxylic acid monomers of step (i) are selected from the group consisting of: lactic acid, glycolic acid, and a mixture thereof.

39. The method according to claim 37, wherein the at least one protected reactive group of step (i) is selected from the group consisting of: ButOxyCarbonyl-ethanolamine, ButOxyCarbonyl-aminopropanol, and a mixture thereof.

40. The method according to claim 37, wherein the at least partially ionizable amino acid of step (ii) is selected from the group consisting of: N-carboxyamino acid anhydrides, amino acid precursor N-carboxyamino acid anhydrides, and a mixture thereof.

41. The method according to claim 40, wherein the at least partially ionizable amino acid is amino acid precursor N-carboxyamino acid anhydrides.

42. The method according to claim 37, wherein the amino acid precursor N-carboxyamino acid anhydrides are deprotected to obtain one or more polyamino acid blocks.

43. The method according to claim 37, wherein the at least one organic solvent of step (ii) is selected from the group consisting of: N-methylpyrrolidone, dimethylformamide, dimethyl sulfoxide, dimethylacetamide, pyrrolidone, dichloromethane, and a mixture thereof.

44. The method according to claim 37, wherein the at least one deprotected α-hydroxycarboxylic acid polymer block of step (i) is added to the poly amino acid block polymerization medium of step (ii) before the step (ii) polymerization under normal atmospheric pressure and at a temperature between 20° and 120° C.

45. The method according to claim 37, further comprising at least one hydrophilic active principle.

46. The method according to claim 45, wherein the at least one hydrophilic active principle is in a solid state.

47. The method according to claim 45, wherein the at least one hydrophilic active principle is selected from the group consisting of: vaccines, peptides, proteins, hemoglobins, cytochromes, albumins, interferons, cytokines, antigens, antibodies, erythropoietin, insulin, growth hormones, factors VIII and IX, interleukins, hematopoiesis-stimulating factors, polysaccharides, heparin, nucleic acids, anti-cancer non-peptido-protein molecules, anthracyclins, taxoids, and mixtures thereof.

48. A pharmaceutical, nutritional, plant-care or cosmetic specialty product created according to the method of claim 37.

49. The method according to claim 37, wherein at least one intermediate product comprising α-hydroxycarboxylic acid—polyamino acid is formed.

50. The method according to claim 49, wherein the at least one intermediate product is selected from the group consisting of: polylactic copolymers, glycolic-polymino acid copolymers, and a mixture thereof.

51. The method according to claim 37, further comprising the steps of:
(iv) precipitating the block copolymer of step (iii) to form a pulverulent solid; and
(v) dissolving the precipitated block copolymer of step (iv) and bringing the block copolymer into contact with a liquid to form a suspension, wherein the liquid contains at least one non-solvent having a pH such that the amino acids of the precipitated block copolymer are at least partially ionized.

52. The method according to claim 51, wherein the at least one non-solvent of step (v) is water.

53. The method according to claim 51, wherein at least one hydrophilic active principle is associated with the block copolymer.

54. The method according to claim 51, wherein the at least one hydrophilic active principle is selected from the group consisting of: vaccines, peptides, proteins, hemoglobins, cytochromes, albumins, interferons, cytokines, antigens, antibodies, erythropoietin, insulin, growth hormones, factors VIII and IX, interleukins, hematopoiesis-stimulating factors, polysaccharides, heparin, nucleic acids, anti-cancer non-peptido-protein molecules, anthracyclins, taxoids, and mixtures thereof.

55. The method according to claim 51, wherein the method further comprises:
(vi) purifying the suspension of step (v).

56. The method according to claim 51, wherein the method further comprises:
(vi) concentrating the suspension of step (v).

57. The method according to claim 51, wherein the method further comprises:
(vi) separating the liquid medium of the suspension of step (v) from the pulverulent solid comprising the particles.

58. A pharmaceutical, nutritional, plant-care or cosmetic specialty product created by the method of claim 51.
